(19) United States
(12) Patent Application Publication
(10) Pub. No.: US 2011/0002949 A1
(43) Pub. Date: Jan. 6, 2011

(54) VACCINE FOR THE TREATMENT OF ALZHEIMER'S DISEASE

(76) Inventors: Mary J. Savage, Chester Springs, PA (US); Gene G. Kinney, Collegeville, PA (US); Xiaoping Liang, Collegeville, PA (US); Michael Citron, Chester Spring, PA (US); Laura B. Rosen, Wayne, PA (US)

Correspondence Address: MERCK, P O BOX 2000, RAHWAY, NJ, 07065-0907, US

(21) Appl. No.: 12/496,823

(22) Filed: Jul. 2, 2009

Related U.S. Application Data
(60) Provisional application No. 61/134,224, filed on Jul. 8, 2008.

(51) Int. Cl. A61K 39/00; G01N 33/53

(52) U.S. Cl. 424/184.1; 435/7.1

(57) ABSTRACT

The invention provides a method for the treatment of a patient having a more severe form of Alzheimer's disease (AD), where the severe form of AD is characterized by pathogenic deposits of amyloid beta peptide (Aβ), comprising the administration of an immunogenic fragment of Aβ capable of inducing an immune response in the form of antibodies to specific to the pathogenic deposits of Aβ and, in particular, to neurotoxic forms of Aβ including N-terminally truncated forms of Aβ. The invention further provides a method for selecting a suitable immunogenic fragment of Aβ for the treatment of a more severe form of AD.

### FIELD OF THE INVENTION

The present invention relates to compositions and methods for the prevention and treatment of amyloidogenic diseases and, in particular, Alzheimer's disease.

### BACKGROUND OF THE INVENTION

Alzheimer's disease (AD) is characterized by progressive memory impairment and cognitive decline. Its hallmark pathological lesions are amyloid deposits (senile plaques), neurofibrillary tangles and neuronal loss in specific brain regions. Amyloid deposits are composed of amyloid beta peptides (Aβ) of 40 to 43 amino acid residues, which are the proteolytic products of amyloid precursor protein (Aβ P). Neurofibrillary tangles are the intracellular filamentous aggregates of hyperphosphorylated tau proteins (Selkoe, Science, 275: 630-631, 1997).

The pathogenesis of AD has not been fully understood, but it is expected to be a multi-factored event. Accumulation and aggregation of Aβ in brain tissue is believed to play a pivotal role in the disease process, also know as the amyloid cascade hypothesis (Golde, Brain Pathol., 15: 84-87, 1995). According to this hypothesis, Aβ, particularly Aβ42, is prone to form various forms of aggregates, ranging from small oligomers to large, elongated proto-fibril structures. These aggregates are neurotoxic and are believed to be responsible for the synaptic pathology associated with the memory loss and cognition decline in the early stage of the disease (Klein et al., Neurobiol. Aging, 25: 569-580, 2004). A recent publication suggests that reduction of Aβ in a triple transgenic mouse model also prevents intracellular tau deposition (Oddo et al., Proc. Neuron, 43:321-332, 2004). This finding suggests that extracellular amyloid deposition may be causative for subsequent neurofibrillary tangle formation, which may in turn lead to neuronal loss.

Immunization of APP transgenic mice with Aβ antigen can reduce the brain Aβ deposits and mitigate disease progression. This was first reported by Shenk et al., Nature, 400: 173-177, 1999, and has now been corroborated by a large number of studies involving different transgenic animal models, various active vaccines as well as passive immunization with Aβ specific monoclonal antibodies (Bard et al., Nature Med, 6: 916-919, 2000; Janus et al., Nature, 408: 979-982, 2000; Morgan et al., Nature, 408: 982-985, 2000; DeMattos et al., Proc. Natl. Acad. Sci., 98: 8850-8855, 2001; Bacskai et al., J. Neurosci., 22: 7873-7878, 2002; Wilcock et al., J. Neurosci., 23: 3745-3751, 2003). Consistent with the animal data, three published evaluations of postmortem human brain tissues from patients who had previously received active immunization with a pre-aggregated Aβ1-42 peptide as an immunogen (AN1792, Betabloc) showed regional clearance of senile plaques (Nicoll et al, Nature Med., 9: 448-452, 2003; Ferrer et al., Brain Pathol., 14: 11-20, 2004; Masliah et al, Neurology, 64: 129-131, 2005). This data collectively indicates that vaccines that effectively elicit antibody responses to Aβ antigens are efficacious against the pathological senile plaques found in AD. However, the mechanism of vaccine or antibody efficacy remains to be defined.

The most advanced study to use an active immunization approach to treat AD has been a Phase II trial using AN1792 (Betabloc) co-administered with the adjuvant, QS-21™ (Antigenics, New York, N.Y.). In January 2002, this study was terminated when four patients showed symptoms consistent with meningoencephalitis (Senior, Lancet Neurol., 1: 3, 2002). Ultimately, 18 of 298 treated patients developed signs of meningoencephalitis (Orgogozo et al, Neurology, 61: 46-54, 2003). There was no correlation between encephalitis and antibody titer and it has been reported that the likely causative mechanism for this effect was activation of T-cells to the self-immunogen, particularly the mid- and carboxy-terminal portion of the Aβ42 (Monsonego et al., J. Clin. Invest., 112: 415-422, 2003). In support of this conclusion, postmortem examination of brain tissue from two vaccine recipients that developed encephalitis revealed substantial meningeal infiltration of CD4⁺ T cells in one patient (Nicoll et al., Nature Med., 9: 448-452, 2003) and CD4⁺, CD8⁺, CD3⁺, CD5⁺, CD7⁺ T cells in the other (Ferrer et al., Brain Pathol., 14: 11-20, 2004). Based in part on these findings, several clinical trials have been initiated with an active anti-Aβ vaccine based on the notion that targeting the N-terminus of Aβ, for example, Aβ1-7 and Aβ1-6, will provide efficacy devoid of T-cell mediated adverse events.

### SUMMARY OF THE INVENTION

In one embodiment, the invention herein is a method of treating patients having a more severe form of Alzheimer's disease (AD) comprising (i) determining that the patient has a more severe form of AD and (ii) administering an immunogenic fragment of Aβ in an amount effective induce an immune response. A patient having a more severe form of AD is selected from the group consisting of an individual with an Mini-Mental State Exam (MMSE) score of 20 or less, an individual with an Alzheimer's Disease Assessment Scale-Cognitive (ADAS-Cog) score of 35 or higher, an individual with a Global Deterioration Scale (GDS) score of stage 5 or higher, an individual with a Clinical Dementia Rating-Sum of Boxes (CDR-SB) score of 2 or higher, an individual who is under 60-64 years of age and presents with symptoms of AD, or an individual diagnosed after genetic screening to have early onset Alzheimer's disease (EOAD) or a familial form of AD. The immunogenic fragment of Aβ comprises a multivalent vaccine comprising multiple, non-contiguous and non-identical immunogenic fragments of Aβ, each have at least one antigenic determinant and lacking a T-cell epitope. In another embodiment, the multivalent vaccine comprises Aβ3-10 and Aβ21-28 connected by a lysine scaffold. The multivalent vaccine further comprises a carrier conjugated to the Aβ peptide fragments and may be optionally administered with an adjuvant.

In another embodiment, the invention herein is a method of selecting an immunogenic fragment of Aβ for use as a vaccine construct suitable for the treatment of patients having a more severe form of Alzheimer's disease (AD) comprising: (i) administering a test immunogenic fragment of Aβ to an animal in an amount effective to induce an immune response; and (ii) evaluating anti-sera from the immunized animal for cross-reactivity to N-terminally truncated forms of Aβ; where a suitable vaccine construct would be selected as one capable of inducing an immune response in the form of antibodies specific to one or more N-terminally truncated forms of Aβ. The N-terminal truncated form of Aβ is selected from the group consisting of Aβx-42, pGlu-Aβ3-40, pGlu-Aβ3-42, pGlu-Aβ11-40, and pGlu-Aβ11-42, where x corresponds to residue 2 to 17 of naturally occurring Aβ.

### BRIEF DESCRIPTION OF THE DRAWINGS

FIG. 1 represents antibodies detected from a serial dilution of antisera from animals immunized with a peptide conjugate of Aβ1-8 (MOVC1-8) conjugated to KLH as a carrier and administered with ISCOMATRIX®.

FIG. 2 represents antibodies detected from a serial dilution of antisera from animals immunized with a multivalent vaccine (Aβ33-10/Aβ21-28) (MVC) conjugated to OMPC as a carrier and administered with ISCOMATRIX®.

### DETAILED DESCRIPTION OF THE INVENTION

The term “8-mer” means an eight amino acid peptide which corresponds to a fragment of Aβ, an analog of a natural Aβ peptide or a peptide mimetic. One or more 8-mers may be combined with at least one space to form a multivalent linear peptide or to form a multivalent branched MAβ.

The term “Aβ conjugate” means an 8-mer or immunogenic fragment of Aβ that is chemically or biologically linked to a carrier, such as keyhole limpet hemocyanin or the outer membrane protein complex of Nesseria meningitidis (OMPC).

The term “Aβ peptide” means any of the synthetic (as compared to naturally occurring amyloid beta peptides (Aβ) Aβ peptides used herein in a vaccine construct, including, but not limited to, linear 8-mers, multivalent linear peptides with at least one spacer and multivalent branched multiple antigenic peptides (MAPs).

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. T-cell epitopes consist of peptides which are capable of forming complexes with host MHC molecules. T-cell epitopes for human MHC class I molecules, which are responsible for induction of CD8⁺T-cell responses, generally comprise 9 to 11 amino acid residues, while epitopes for human MHC class II molecules, which are responsible for CD4⁺T-cell responses, typically comprise 12 or more amino acid residues (Bjorkman et al. Nature 329:506-512, 1987; Madden et al. Cell 75:693-708; Batalia and Collins; Engelhard Annu Rev Immunol., 12: 181-207-622. 1995; Madden, Annu Rev Immunol., 13:587-622. 1995). Unlike T cells, B cells are capable of recognizing peptides as small as 4 amino acids in length. It is the T-cell epitope/MHC complexes that are recognized by T-cell receptors leading to T cell activation.

The term “multivalent peptide” refers to peptides having more than one antigenic determinant.

The term “multivalent vaccine” or “MVC” means a vaccine construct composed of multiple Aβ peptides, each having an antigenic determinant and lacking a T cell epitope. In one embodiment, the multivalent vaccine comprises two non-contiguous, non-identical, immunogenic fragments of Aβ, for example, Aβ3-10 and Aβ21-28, each lacking a T-cell epitope.

The term “immunogenic fragment of Aβ” or “immunogenic fragment of Aβ lacking a T-cell epitope” means an 8-mer or an Aβ fragment that is capable of inducing an immune response in the form of antibodies to Aβ, but which response does not include a T-cell response to the self antigen, Aβ.

The term “immunological” or “immune” or “immunogenic” response refers to the development of a humoral (antibody mediated) and/or a cellular (mediated by antigen-specific T cells or their secretion products) response directed against an antigen in a vertebrate individual. Such a response can be an active response induced by administration of an immunogen or a passive response induced by administration of an antibody.

The term “a more severe form of AD” refers to a patient having any form of AD that is associated with a more advanced form of neuronal degeneration, as compared to an age-control non-AD patient, or who exhibits a more advanced clinical pathology. Such patients include, but are not limited to, an individual with an Mini-Mental State Exam (MMSE) score of 20 or less, an individual with an Alzheimer's Disease Assessment Scale- Cognitive (ADAS-Cog) score of 35 or higher, an individual with a Global Deterioration Scale (GDS) score of stage 5 or higher, an individual with a Clinical Dementia Rating-Sum of Boxes (CDR-SB) score of 2 or higher, an individual who is under 60-64 years of age and presents with symptoms of AD, or an individual diagnosed after genetic screening to have early onset Alzheimer's disease (EOAD) or a familial form of AD, particular those associated with a PS-1 mutation, or a patient having a form of AD characterized by pathogenic deposits of Aβ.

The term “pathogenic deposits of amyloid beta peptide (Aβ)” or “pathogenic deposits of Aβ” means plaque deposits comprising neurotoxic forms of Aβ, for example Aβ42, or N-terminally or C-terminally truncated forms of Aβ known to be associated with more neuronal degeneration or more severe clinical phenotype. Such forms of Aβ include, but are not limited to, Aβ40, Aβ42, N-terminally truncated forms of Aβ, for example, Aβx-42, where x corresponds to residues 2-17 of naturally occurring Aβ, and truncated forms of Aβ modified by cyclization of the terminal amino acids, for example, cyclization of the N-terminal glutamates, pGluAβ3-42 or pGluAβ11-42.

The term “antibodies specific to a pathogenic Aβ deposit” refers to an antibody that is cross-reactive with a neurotoxic form of Aβ, including full length Aβ40 or Aβ42, N-terminally truncated forms of Aβ or N-terminally or C-terminally truncated forms of Aβ having modifications at the terminal amino acid, such as pGluAβ3-42 or pGluAβ11-42.

The term “pharmaceutical composition” means a chemical or biological composition suitable for administration to a mammalian individual. As used herein, it refers to a composition comprising 8-mers, immunogenic fragments of Aβ and Aβ conjugates described herein to be administered optionally with or without an adjuvant.

### Pathogenic Deposits of Amyloid Beta Peptide (Aβ)

Increasing evidence suggests that the Aβ deposited in the brains of AD patients is not homogenous in structure (Saido et al., Neuroscience Letters, 215:173-176, 1996). In addition to multiple forms of the full length amyloid beta peptide (Aβ) Aβ40 and Aβ42, multiple truncated forms of Aβ, having modifications at the N-terminal and C-terminal ends of the peptide, have been detected (Russo et al., FEBS Letters, 409: 411-416, 1997; Saido 1996). Increasingly it is thought that these truncated forms of Aβ are critical in AD development (Piccini et al., J. Biol. Chem., 280 (40): 34186-34192, 2005). Among these truncated forms of Aβ, N-terminally truncated peptides starting with pyroglutamyl at residues Glu3 or Glu1I predominate (Russo, 1997). The pGlu3 form (Aβ3(pE)-42) is especially prevalent, comprising about 50% of total Aβ (Youssef et al., Neurobiol. Aging, 29: 1319-1333, 2008).

These N-terminally truncated forms have been found to accumulate early in the brains of patients diagnosed with sporadic AD, in early onset familial AD (EOAD) patients, most particularly those having presenilin-1 (PS-1) mutations, and in patients with Down's Syndrome (DS) (Russo et al., FEBS Lett., 409: 411-416, 1997; Saido et al., Neurosci. Lett., 215: 173-176, 1996; Tekirian et al., J. Neuropathol. Ex. Neurol., 57: 76-94, 1998). Individuals with EOAD driven by PS-1 mutations develop disease symptoms typically before 60-64 years of age as compared to those with sporadic, late onset AD (LOAD) harboring no mutations. In addition, patients with Down's syndrome (DS) also develop EOAD due to their extra copy of chromosome 21, the same chromosome on which genes associated with some of the inherited forms of AD are located, leading to 30% more Aβ P and increased Aβ production. Familial Danish dementia is another form of early onset dementia characterized by a large, almost exclusive fraction of pyroGlu, N-terminally modified Aβ (Tomidokoro, et al., J. Biol. Chem., 280 (44): 36883-36894, 2005). Individuals presenting with EOAD due to PS-1 mutations or DS harbor significantly more pGluAβ3-42 in their brain as compared to LOAD (Russo, 1997; Russo et al., Nature, 405: 531-532, 2000; Russo et al., Neurobiol. Dis., 8: 173-180, 2001; Hosoda et al., J. Neuropathol. Exp. Neurol., 57: 1089-1095, 1998). More importantly, patients having a greater proportion of the N-terminally truncated forms as determined from postmortem tissue analysis, particularly the predominant pGluAβ3-42 form, get more severe disease, both in terms of the degree of neuronal degeneration and the severity of the clinical pathology (Russo, 1997; Russo 2000).

An assessment of an individual for AD or dementia would generally include some form of mental or cognitive assessment, which could be carried out by various methods including the Alzheimer's Disease Assessment Scale-Cognitive (ADAS-Cog), the Global Deterioration Scale (GDS), the Clinical Dementia Rating—summary of boxes (CDR-SB), or more typically a Mini-Mental State Exam (MMSE). MMSE scores have a maximum of 30, with scores generally classified as mild (21-26), moderate (15-20) and severe (14 or less). Scores for ADAS-Cog range from 0 (best possible) to 70 (worse possible), with scores of around 23 being the cutoff for mild impairment and scores of about 35 or higher correlating with moderate and above impairment. Scores for CDR have a maximum of 4, with scores classified as normal (0), mild (0.5-1), moderate (2), and severe (3-4). Similarly, scores for GDS range from stage 1 (best) to stage 7 (worst), with grade 4 being comparable to an ADAS-Cog score of about 22.5 for mild impairment and stage 5 being comparable to an ADAS-Cog score of about 35 for moderate impairment. See, Folstein et al., J. Psychiat. Res., 12: 189-198, 1975, for a general discussion of MMSE in relationship to AD and dementia. See Doraiswamy et al., Neurology, 48 (6): 1511-1517, 1997, for a comparison of ADAS-Cog, MMSE and GDS scoring and validity. ADAS-Cog and MMSE have been generally accepted for use in assessment of efficacy in clinical trials. Another factor to consider would be the individual's family history, that is, whether another (or multiple) closely related family member had a form of AD considered to be severe. To confirm the presence of EOAD due to FAD mutations, one could perform sequence analysis on genomic DNA from the patient's white blood cells (Finckh, et al., Am. J. Hum. Genet., 66: 110-117, 2000). Accordingly, individuals presenting with an early, aggressive form of AD or dementia, such as EOAD or FAD, particularly those under 60-64 years of age, or those scoring 20 or less on a MMSE would be considered to have a more severe form of AD and expected to have plaques characterized by pathogenic amyloid deposits, including the N-terminally truncated forms, and would be candidates for the multivalent vaccine herein.

Applicants herein have found that a vaccine construct comprising multiple immunogenic fragments of Aβ provides a more effective means to treat AD patients having a more severe form of AD associated with N-terminally truncated forms of Aβ. The multivalent vaccine is a broad spectrum vaccine in that it is capable of treating patients having forms of AD with plaques comprised not only of the full-length form of Aβ associated with AD, but also N-terminally truncated forms of Aβ. The multivalent vaccine of the invention is capable of cross-reacting with multiple and more forms of neurotoxic Aβ, particularly with respect to N-terminally truncated forms. Applicants herein show for the first time that a multivalent vaccine, comprising multiple non-contiguous, non-identical immunogenic fragments of Aβ, lacking a T-cell epitope, can be more effectively employed to treat AD and, in particular, those patients having species of Aβ known to be correlated with more severe forms of the disease in terms of neuronal degeneration and clinical pathology.

### Vaccine Constructs to Treat a More Severe Form of AD

Applicants herein have surprisingly found that a vaccine construct, comprising multiple immunogenic fragments of Aβ lacking a T-cell epitope, referred to herein as a multivalent vaccine, can provide a broad spectrum vaccine to treat patients having a more severe form of AD and specifically those having pathogenic deposits of Aβ comprising an N-terminally truncated form of Aβ. Inasmuch as other anti-Aβ vaccine constructs reported in the literature appear to be directed to a single immunogenic fragment of Aβ, the invention herein provides an advantage and a more effective vaccine for targeting those forms of AD known to be correlated with the presence of the N-terminally truncated forms of Aβ.

In a related co-pending application Applicants have described compositions and methods of the use of peptide conjugates comprising immunogenic fragments of Aβ, lacking a T-cell epitope, and that are capable of inducing a beneficial immune response in the form of antibodies to Aβ (PCT/US 2006/016481, WO 2006/121656; U.S. Ser. No. 11/919,897, US 2009-0098155, the teachings of which are incorporated herein as if set forth at length) to treat AD. The vaccine compositions therein are composed of immunogenic fragments of Aβ which were limited in size to eight amino acids (8-mers) and were designed to remove any potential C-terminal T-cell epitope anchor residues. The immunogenic fragment of Aβ can be an 8-mer linear peptide, a multivalent linear Aβ conjugate having at least one PEG spacer or a multivalent branched multiple antigenic peptide (MAβ). In a preferred embodiment the vaccine construct is a branched MAβ comprising Aβ3-10 and Aβ21-28 connected on a lysine scaffold.

The vaccine constructs for use in an active immunization regime to treat AD therein can be administered in the form of a pharmaceutical composition, in which the immunogenic fragment of MAβ can be linked either chemically or biologically to a carrier, such as serum albumins, keyhole limpet hemocyanin (KLH), immunoglobulin molecules, ovalbumin, tetanus toxoid protein, or a toxoid from other pathogenic bacteria, such as diphtheria, E. coli, cholera, or H. pylori, or an attenuated toxin derivative. In a preferred embodiment the carrier is the outer membrane protein complex of Neisseria meningitidis (OMPC).

The vaccine constructs for use in an active immunization regime to treat AD therein may be administered with an adjuvant, such as aluminum salts (alum), a lipid, such as 3-de-O-acylated monophosphoryl lipid A (3D-MPL) or a saponin-based adjuvant. In a preferred embodiment the adjuvant is a saponin-based adjuvant, ISCOMATRIX® (CSL Ltd, Parkville, Australia).

Applicants herein have surprisingly found that a preferred embodiment of the peptide conjugate therein, a multivalent vaccine comprising a branched MAβ of Aβ3-10 and Aβ21-28 connected with a lysine scaffold and conjugated to OMPC, now provides a broad spectrum active vaccine for the treatment of AD. The structure for this multivalent vaccine (MVC) is as follows:

| | |
|---|---|
| Ac-EFRHDSGY(Aha)-Lys-Lys-(BrAc)-NH₂ | (SEQ ID NO: 1) |
| Ac-AEDVGSNK(Aha) | (SEQ ID NO: 2) |

wherein “Aha” represents 6-aminohexanoic acid and “BrAc” represents bromoacetyl.

Applicants have shown herein that this broad spectrum MVC offers an advantage versus other active vaccine approaches currently undergoing clinical assessment. This multivalent vaccine has not only been shown to provide an immune response, in the form of antibodies that specifically cross-react with multiple forms of Aβ and, in particular the N-terminally truncated forms of Aβ associated with the more severe forms of AD, it provides a stronger immune response in that Aβ1-8 vaccine did not produce any immune response to Aβx-42, when x≧3. As such, the multivalent vaccine herein is capable of providing better immunogenicity, i.e. a broader spectrum of response, to the N-terminally truncated forms of Aβ than other active vaccines under clinical consideration.

### Therapeutic Agents for the Treatment of a More Severe Form of AD

Applicants immunized guinea pigs with a multivalent vaccine construct, a branched MAβ comprising Aβ3-10 and Aβ21-28 connect via a lysine scaffold (herein referred to as a multivalent vaccine construct—MVC) conjugated to a carrier (OMPC) and administered with a saponin-based adjuvant, ISCOMATRIX®. The immunized animals generated an immune response in the form of polyclonal antibodies. Serum was drawn from the animals and the antisera was serially diluted and tested for cross-reactivity against numerous forms of Aβ including full length Aβ40 and Aβ42, and the N-terminal truncated forms of Aβ listed in Table 1.

Similarly, Applicants immunized guinea pigs with a synthetic monovalent Aβ peptide corresponding to amino acid residues 1-8 of naturally occurring Aβ (herein referred to as a monovalent vaccine construct—MOVC1-8) conjugated to a carrier (KLH) and administered with a saponin-based adjuvant, ISCOMATRIX®. Upon information and belief, it is believed that other active vaccines currently undergoing clinical evaluation employ similar monovalent vaccine constructs corresponding to Aβ1-7 and Aβ1-6 conjugated to CRM197 or a VLP, respectively. The Aβ1-7/CRM197 vaccine construct is believed to be administered with a saponin-based adjuvant, QS-21, while the Aβ1-6NVLP construct is not administered with an adjuvant.

Active vaccines presently in clinical trials for AD include the N-terminal, residue 1, of the Aβ sequence and are 6-7 amino acids in length. In contrast thereto, the MVC utilized by Applicants comprises an immunogenic fragment of Aβ corresponding to Aβ residue 3 and ending at residue 10 and a second immunogenic fragment of Aβ corresponding to residue 21 and ending at residue 28. As demonstrated herein, this MVC recognizes more N-terminally truncated forms of Aβ as compared to the other active vaccine approaches employing peptides starting at Aβ residue 1. Without wishing to be bound by any theory, it is believed that other multivalent vaccine constructs described in WO 2006/121656 will perform with similar specificity. Those of ordinary skill in the art would recognize and appreciate that the use of a multivalent 8-mer antigens will produce a response that is representative of any fragment length that could be incorporated into a vaccine construct as described herein, provided that the fragment length is capable of producing a desired polyclonal immune response while not stimulating an antigen directed T-cell response. Thus, the invention described herein could, in alternate embodiments, comprise Aβ fragments including, but not limited to, 7-mers, 6-mers, 5-mers and 4-mers.

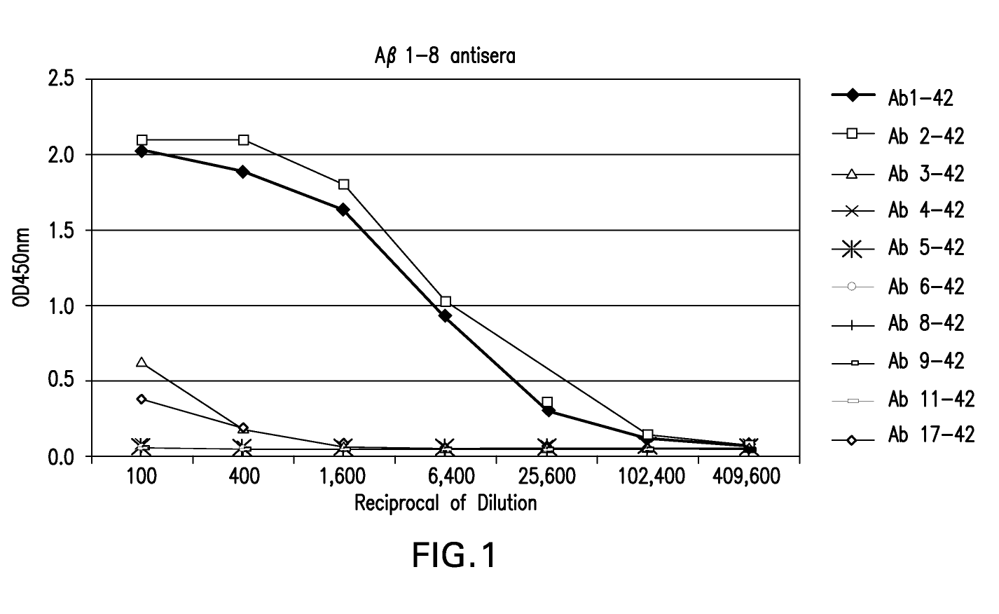

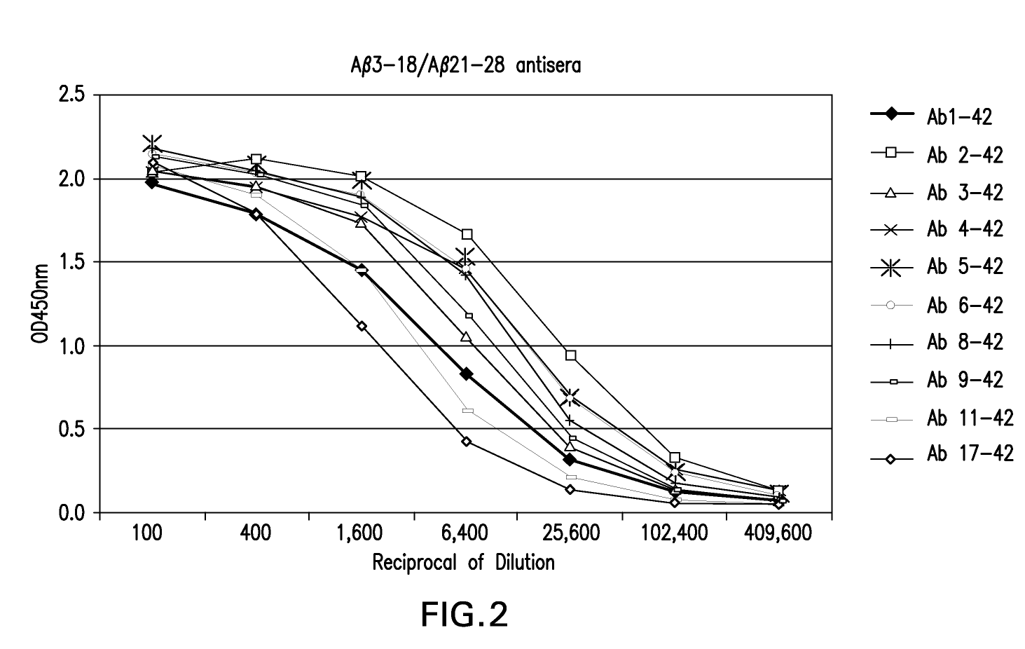

Prior to undertaking the experiments herein, Applicants sought to predict based on the composition of the vaccine constructs, which forms of Aβ, either full length or N-terminal truncated forms, with which the antisera from the vaccinated animals would cross-react. These predictions are shown in Table 1 as compared to the actual species with which the antisera from the multivalent vaccine construct (Aβ3-10/Aβ21-28) (MVC) and the monovalent vaccine construct Aβ1-8 (MoVC1-8) cross-reacted. The degree of cross-reactivity for each form of Aβ is also shown in FIGS. 1 and 2. As is evident from FIGS. 1 and 2, not only did the MVC described herein recognize a greater number of N-terminally modified Aβ peptides as compared to the MoVC1-8 construct, it also had greater cross-reactivity with the forms most associated with the severest forms of the disease, and specifically pGlu3 Aβ3-42. Most succinctly, this data demonstrates that the multivalent vaccine is likely to induce antibodies which are capable of binding to truncated forms starting at the free N-terminus (Aβ1-x) compared to vaccines comprised of peptides equal to or less than eight amino acids.

TABLE 1

| | Multivalent | | Monovalent | |
|---|---|---|---|---|
| | Vaccine (MVC) | | Vaccine (MoVC1-8) | |
| | (Aβ3-10/Aβ21-28) | | (Aβ1-8) | |
| N-terminally | Predicted | | Predicted | |
| truncated Aβ | cross- | Actual cross- | cross- | Actual cross- |
| peptide | reactivity | reactivity | reactivity | reactivity |
| Aβ(1-42) | + | + | + | + |
| Aβ(2-42) | + | + | + | + |
| [pGlu] Aβ (3-42) | + | + | + | − |
| Aβ(4-42) | + | + | + | − |
| Aβ(5-42) | + | + | + | − |
| Aβ(6-42) | + | + | + | − |
| Aβ(8-42) | + | + | − | − |
| Aβ(9-42) | + | + | − | − |
| [pGlu] Aβ (11-42) | + | + | − | − |
| Aβ(17-42) | + | + | − | − |

While several N-terminal truncated Aβ peptides are more toxic or equally toxic as compared to peptides starting at residue 1, one peptide in particular is orders of magnitude more toxic; Aβ starting at residue 3, and modified by glutaminyl cyclase, termed pyroglu3 Aβ (pGlu3 Aβ3-42). The predominance of this truncated form of Aβ has been shown to be directly proportional to the intensity of neuronal degeneration and the severity of the clinical phenotype (Youssef et al., Neurobiology of Aging, 29:1319-1333, 2008). Applicants have demonstrated that serum from mammals generated following immunization with a monovalent vaccine (MoVC1-8) does not interact with the toxic species pGlu3Aβ3-42. One skilled in the art will also appreciate and recognize that the shorter peptide immunogens currently being used in clinical trials (Aβ1-7 and Aβ1-6) will also fail to recognize the pGlu3Aβ3-42 form. Immunization with other multivalent vaccines, such as those comprising Aβ3-8 and Aβ21-28 would also be expected to recognize N-terminally truncated forms of Aβ, as well as those ending at a variety of carboxy termini, including -38,-40 and -42, which are the most common C-terminal truncated forms.

As demonstrated by this cross-reactivity, the invention claimed addresses the clinical problem of the more severe forms of AD resulting from the presence of multiple N-terminally truncated forms of Aβ present in the plaques of Alzheimer's diseased brains. Without wishing to be bound by any theory, one possible limitation to AD vaccines employing peptides that include the N-terminal, residue 1, and are limited to six or seven amino acids in length, such as those currently undergoing clinical evaluation, is that it is more likely that not that they would produce an immune response only to these limited forms of Aβ in vivo, specifically, only to those forms of Aβ that included the N-terminal residue. In a preferred form, it would be desirable for the AD vaccine to induce an immune response, in the form of antibodies that specifically cross-react, to all N-terminally truncated forms of Aβ in addition to forms including residue 1. Inasmuch as the N-terminally truncated forms of Aβ are correlated with more severe forms of AD, this broader recognition would be expected to allow for use in a less restricted clinical population. Thus, one skilled in the art would appreciate and recognize that the invention claimed herein, the use of a multivalent vaccine, exemplified using a vaccine comprised of immunogenic fragments of Aβ corresponding to Aβ3-10 and Aβ21-28, that recognizes all N-terminal truncated forms of Aβ, will enable a more effective treatment of AD patients having a more severe form of AD than that provided by a monovalent vaccine that only recognizes those forms of Aβ that include the N-terminal, residue 1. Following this rationale, patients immunized with either Aβ1-6 or Aβ31-7 will not be protected to the same degree as those vaccinated with a MVC, and especially will not be protected from the toxic effects of the N-terminally truncated forms of Aβ.

### Treatment Regimes

Effective doses of the multivalent vaccine herein for the therapeutic treatment of a more severe form of AD and other amyloid diseases will vary depending upon many factors including, but not limited to, means of administration, target site, physiological state of the patient, other medications administered and whether treatment is a therapeutic, i.e. after on-set of disease symptoms, or prophylactic, i.e. to prevent the on-set of disease symptoms. In a preferred embodiment the patient is human and the therapeutic agent is to be administered by injection.

The amount of immunogen or therapeutic agent to be employed will also depend on whether an adjuvant is to be administered either concomitantly or sequentially, with higher doses being employed in the absence of an adjuvant.

The amount of an immunogen or therapeutic agent to be administered will vary, but amounts ranging from 0.5-50 μg of peptide (based on the Aβ peptide content) per injection are considered for human use. Those skilled in the art would know how to formulate compositions comprising antigens of the type described herein.

The administration regimen would consist of a primary immunization followed by booster injections at set intervals. The intervals between the primary immunization and the booster immunization, the intervals between the booster injections, and the number of booster immunizations will depend on the antibody titers and duration elicited by the vaccine. It will also depend on the functional efficacy of the antibody responses, namely, levels of antibody titers required to prevent AD development or exerting therapeutic effects in AD patients. A typical regimen will consist of an initial set of injections at 1, 2 and 6 months. Another regimen will consist of initial injections at 1 and 2 months. For either regimen, booster injections will be given either every six months or yearly, depending on the antibody titers and durations. An administration regimen can also be on an as-needed basis as determined by the monitoring of immune responses in the patient.

### Selection of Immunogenic Fragments of Aβ for Use in Treating More Severe Forms of AD

One skilled in the art will appreciate that this invention also provides a method to identify new vaccines capable of producing an immune response in the form of antibodies that broadly and specifically cross-react to N-terminally or C-terminally truncated forms of Aβ. In one embodiment, a test immunogenic fragment of Aβ, i.e. a test vaccine construct, would be used to immunize an animal, such as a guinea pig or other rodent. The vaccine construct may further comprise a conjugate in which the peptide construct is conjugated to a protein carrier. The vaccine construct may also be optionally administered with an adjuvant to modify the nature of and/or the magnitude of the immune response. The anti-sera from the immunized animal would be evaluated for the presence of polyclonal antibodies generated by vaccination with the construct that specifically cross-react with one or more truncated forms of Aβ, including, but not limited to, pGluAβ3-42, pGluAβ11-42, pGluAβ3-40 or pGluAβ11-40, as measured by ELISA or other format. Vaccine constructs producing broad and specific cross-reactivity would be selected for use in treating patients with a more severe form of AD or related disorders characterized by truncated forms of Aβ. In that disease severity is directly proportional to the presence of N-terminally truncated species of Aβ, one of ordinary skill in the art would recognize and appreciate that patients exhibiting a more severe form of AD, identified based on their by cognitive scores, genetic screening or clinical observation, would be particularly responsive to treatment.

### EXAMPLE 1

### A. Preparation of Peptides and Immunogens

The peptides used herein were, with the exception of Aβ42, were purchased from Anaspec, San Jose, Calif. A listing of these peptides is given in Table 2. Aβ42 was prepared as shown in Example 1.B.

TABLE 2

| | |
|---|---|
| β-Amyloid(1-42) | Example 1.B |
| DAEFRHDSGYEVHHQKLVFFAEDVGSNKG | |
| AIIGLMVGGVVIA | |
| (SEQ ID NO: 3) | |
| β-Amyloid(2-42) | Anaspec, San Jose, CA |
| AEFRHDSGYEVHHQKLVFFAEDVGSNKGA | Cat # 29909-01 |
| IIGLMVGGVVIA | |
| (SEQ ID NO: 4) | |
| [pGlu]-β-Amyloid(3-42) | |
| PyrE-FRHDSGYEVHHQKLVFFAEDVGSN | Anaspec, San Jose, CA |
| KGAIIGLMVGGVVIA | Cat # 29907-01 |
| (SEQ ID NO: 5) | |
| β-Amyloid(4-42) | |
| FRHDSGYEVHHQKLVFFAEDVGSNKGAII | Anaspec, San Jose, CA |
| GLMVGGVVIA | Cat # 29908-01 |
| (SEQ ID NO: 6) | |
| β-Amyloid(5-42) | |
| RHDSGYEVHHQKLVFFAEDVGSNKGAIIG | Anaspec, San Jose, CA |
| LMVGGVVIA | Cat # 60087-01 |
| (SEQ ID NO: 7) | |
| β-Amyloid(6-42) | |
| HDSGYEVHHQKLVFFAEDVGSNKGAIIGL | Anaspec, San Jose, CA |
| MVGGVVIA | Cat # 60086-01 |
| (SEQ ID NO: 8) | |
| β-Amyloid(8-42) | |
| SGYEVHHQKLVFFAEDVGSNKGAIIGLMV | Anaspec, San Jose, CA |
| GGVVIA | Cat # 60085-01 |
| (SEQ ID NO: 9) | |
| β-Amyloid(9-42) | |
| GYEVHHQKLVFFAEDVGSNKGAIIGLMVG | Anaspec, San Jose, CA |
| GVVIA | Cat # 60084-01 |
| (SEQ ID NO: 10) | |
| [pGlu]-β-Amyloid(11-42) | |
| PyrE-VHHQKLVFFAEDVGSNKGAIIGLM | Anaspec, San Jose, CA |
| VGGVVIA | Cat # 29903-01 |
| (SEQ ID NO: 11) | |
| β-Amyloid(17-42) | |
| LVFFAEDVGSNKGAIIGLMVGGVVIA | Anaspec, San Jose, CA |
| (SEQ ID NO: 12) | Cat # 22815 |

### B. Preparation of Aβ1-42 and Other Aβ Peptides

Starting with Rink Amide MBHA resin, the Aβ1-42 peptide was prepared by solid-phase synthesis on an automated peptide synthesizer using Fmoc chemistry protocols as supplied by the manufacturer (Applied Biosystems, Foster City, Calif.). Following assembly the resin bound peptide was deprotected and cleaved from the resin using a cocktail of 94.5% trifluoroacetic acid, 2.5% 1,2-ethanedithiol, 1% triisopropylsilane and 2.5% H₂O. Following a two hour treatment the reaction was filtered, concentrated and the resulting oil triturated with ethyl ether. The solid product was filtered, dissolved in 50% acetic acid/H₂O and freeze-dried. Purification of the semi-pure product was achieved by RPHPLC using a 0.1% TFA/H₂O/acetonitrile gradient on a C-18 support. Fractions were evaluated by analytical HPLC. Pure fractions (>98%) were pooled and freeze-dried. Identity was confirmed by amino acid analysis and mass spectral analysis.

All other peptides were synthesized using similar Fmoc chemistry at Anaspec, San Jose Calif.

### C. Preparation of Aβ1-8-KLH Conjugate

The Aβ peptides (8-mers), 2 mg, were suspended in 1 ml of commercial maleimide conjugation buffer (83 mM sodium phosphate, 0.1 M EDTA, 0.9 M NaCl, 0.02% sodium azide, pH 7.2 (Pierce Biotechnology, Rockford, Ill.). A 2 mg sample of commercial maleimide-activated KLH (Pierce Biotechnology, Rockford, Ill.) was added to the peptide and allowed to react at 25° C. for four hours. The conjugate was separated from unreacted peptide and reagents by exhaustive dialysis versus PBS buffer using 100,000 Da dialysis tubing. The amount of peptide incorporated into the conjugate was estimated by amino acid analysis following a 70 hour acid hydrolysis. Peptide concentrations were determined to be between 0.24 and 0.03 mg/ml.

### D. Synthesis of Bromoacetylated Aβ(3-10)(21-28)

Bromoacetylated peptide was prepared by standard t-Boc solid-phase synthesis, using a double coupling protocol for the introduction of amino acids on the Applied Biosystems model 430A automated synthesizer. Following coupling of the carboxyterminal Fmoc-Lys(ivDde)-OH [ivDde=1, (4,4-Dimethyl-2,6-dioxo-cyclohexylidene)-3-methyl-butyl] to MBHA resin the α-amino Fmoc protecting group was removed using piperidine and the synthesis continued with the introduction of t-Boc-Lys(Fmoc)-OH. After deprotection of the t-Boc group the sequence was extended with the following t-Boc protected amino acids: Aha, Y, G, S, D, H, R, F, E and the amino terminus capped by coupling acetic acid on the ABI synthesizer. The side chain lysine Fmoc protecting group was removed with piperidine and the N^{ε} arm of lysine extended on the ABI synthesizer with the introduction of the following protected amino acids: Aha, K, N, S, G, V, D, E, A, and the amino terminus capped by coupling acetic acid. Removal of the ivDde protecting group was by treatment with 5% hydrazine in dimethylformamide for 5 minutes providing the unblocked N^{ε} amino group on the carboxy terminal lysine The N^{ε} amino group was reacted with Bromoacetic anhydride in methylene chloride as the solvent for 30 minutes. Removal of the peptide from the resin support was achieved by treatment with liquid hydrofluoric acid and 10% anisole as a scavenger. The peptides were purified by preparative HPLC on reverse phase C-18 silica columns using a 0.1% TFA/acetonitrile gradient. Identity and homogeneity of the peptides were confirmed by analytical HPLC and mass spectral analysis.

### EXAMPLE 2

### Generation of Guinea Pig Anti-Aβ Peptide Sera

Six to ten week-old female guinea pigs were obtained from Charles River, Inc., Raleigh, N.C. and maintained in the animal facilities of Merck Research Laboratories in accordance with institutional guidelines. All animal experiments were approved by Merck Research Laboratories Institutional Animal Care and Use Committee (IACUC). Aβ peptide conjugates, Aβ1-8 (MoVCAβ1-8)-KLH and Aβ (3-10)(21-28) (MVC)-OMPC, were formulated with 100 μg/ml of ISCOMATRIX® (CSL, Ltd., Parkville, Australia) and 100 μg/ml of ISCOMATRIX® plus 450 μg/ml of Merck aluminum alum, respectively. The final antigen concentrations, based on the peptide content, were 8 μg/ml and 4 μg/ml for Aβ1-8-KLH and Aβ (3-10)(21-28)-OMPC, respectively. Two guinea pigs were immunized with 400 μl of each conjugate intramuscularly twice at four week intervals and blood samples were collected between three and four weeks following the second immunization. Serum samples from each group were pooled and stored at 4° C. until use.

### EXAMPLE 3

### Binding of Guinea Pig Antisera to Various Forms of Various Forms of Aβ Peptides.

Binding activity of guinea pig antisera to the Aβ peptides, full length and N-terminal truncated, were carried out by enzyme-linked immunosorbent assay (ELISA). Ninety-six well plates (Immuno 96 MicroWell™ Plate, ThermoFisher Scientific, Rochester, N.Y.) were coated with 50 μl per well of various Aβ peptides as shown in Table 2 at a concentration of 4 μg per ml in PBS at 4° C. over night. Plates were washed six times with PBS containing 0.05% Tween-20 (PBST) and blocked with 3% skim milk in PBST (milk-PBST). Guinea pig antiserum was prepared in milk-PBST at serial 4-fold dilutions. One hundred μl diluted anti-sera were added to each well and the plates were incubated for two hours at room temperature, followed by three washes with PBST. Fifty μl of HRP-conjugated goat anti-guinea pig secondary (Jackson Immuno Research, West Grove, Pa.) at a 1:5000 dilution in milk-PBST was added per well and then incubated at room temperature for one hour. The plates were washed six times, followed by the addition of 100 μl per well of 3,3′,5,5′-tetramethylbenzidine (TMB) (Virolabs, Chantilly, Va.). After three to five minutes incubation at room temperature the reaction was stopped by adding 100 μl of stop solution (Virolabs, Chantilly, Va.) per well. The plates were read at 450 nm in a VersaMax™ microplate reader (Molecular Devices, Sunnyvale, Calif.).

Results of this assay are shown graphically in FIGS. 1 and 2, evaluating the various Aβ peptides against guinea pig sera to MoVC1-8 and MVC. The graphs use the average absorbance from each test sample, run in triplicate against each peptide.

What is claimed is:

1. A method of treating patients having a more severe form of Alzheimer's disease (AD) comprising (i) determining that the patient has a more severe form of AD and (ii) administering an immunogenic fragment of Aβ in an amount effective to induce an immune response.

2. The method of claim 1 where a patient having a more severe form of AD is selected from the group consisting of an individual with an Mini-Mental State Exam (MMSE) score of 20 or less, an individual with an Alzheimer's Disease Assessment Scale-Cognitive (ADAS-Cog) score of 35 or higher, an individual with a Global Deterioration Scale (GDS) score of stage 5 or higher, an individual with a Clinical Dementia Rating-Sum of Boxes (CDR-SB) score of 2 or higher, an individual who is under 60-64 years of age and presents with symptoms of AD, or an individual diagnosed after genetic screening to have early onset Alzheimer's disease (EOAD) or a familial form of AD.

3. The method of claim 2 wherein the immunogenic fragment of Aβ comprises a multivalent vaccine comprising multiple, non-contiguous immunogenic fragments of Aβ, each lacking a T-cell epitope.

4. The method of claim 3 wherein the multivalent vaccine comprises Aβ3-10 and Aβ21-28 connected via a lysine scaffold.

5. The method of claim 4 wherein the multivalent vaccine further comprises a carrier conjugated to the Aβ immunogenic fragments.

6. The method of claim 5 wherein the multivalent vaccine is administered with an adjuvant.

7. A method of selecting an immunogenic fragment of Aβ for use as a vaccine construct suitable for the treatment of patients having a more severe form of Alzheimer's disease (AD) comprising:
(i) administering a test immunogenic fragment of Aβ to an animal in an amount effective to induce an immune response; and
(ii) evaluating anti-sera from the immunized animal for cross-reactivity to N-terminally truncated forms of Aβ;
where a suitable vaccine construct would be selected as one capable of inducing an immune response in the form of antibodies specific to one or more N-terminally truncated forms of Aβ.

8. The method of claim 7 wherein the N-terminal truncated form of Aβ is selected from the group consisting of Aβx-42, pGlu-Aβ3-40, pGlu-Aβ 3-42, pGlu-Aβ11-40, and pGlu-Aβ11-42, where x corresponds to residue 2 to 17 of naturally occurring Aβ.


SEQUENCE LISTING (12 sequences)

SEQ ID NO: 1 - protein, 8 aa, Artificial Sequence
  sequence is synthesized
EFRHDSGY

SEQ ID NO: 2 - protein, 8 aa, Artificial Sequence
  sequence is synthesized
AEDVGSNK

SEQ ID NO: 3 - protein, 42 aa, Artificial Sequence [sequence omitted]

SEQ ID NO: 4 - protein, 41 aa, Artificial Sequence
  sequence is synthesized
AEFRHDSGYEVHHQKLVFFAEDVGSNKGAIIGLMVGGVVIA

SEQ ID NO: 5 - protein, 40 aa, Artificial Sequence
  sequence is synthesized
XFRHDSGYEVHHQKLVFFAEDVGSNKGAIIGLMVGGVVIA

SEQ ID NO: 6 - protein, 39 aa, Artificial Sequence
  sequence is synthesized
FRHDSGYEVHHQKLVFFAEDVGSNKGAIIGLMVGGVVIA

SEQ ID NO: 7 - protein, 38 aa, Artificial Sequence
  sequence is synthesized
RHDSGYEVHHQKLVFFAEDVGSNKGAIIGLMVGGVVIA

SEQ ID NO: 8 - protein, 37 aa, Artificial Sequence
  sequence is synthesized
HDSGYEVHHQKLVFFAEDVGSNKGAIIGLMVGGVVIA

SEQ ID NO: 9 - protein, 35 aa, Artificial Sequence
  sequence is synthesized
SGYEVHHQKLVFFAEDVGSNKGAIIGLMVGGVVIA

SEQ ID NO: 10 - protein, 34 aa, Artificial Sequence
  sequence is synthesized
GYEVHHQKLVFFAEDVGSNKGAIIGLMVGGVVIA

SEQ ID NO: 11 - protein, 34 aa, Artificial Sequence
  sequence is synthesized
XAAVHHQKLVFFAEDVGSNKGAIIGLMVGGVVIA

SEQ ID NO: 12 - protein, 26 aa, Artificial Sequence
  sequence is synthesized
LVFFAEDVGSNKGAIIGLMVGGVVIA